(19) United States
(12) Patent Application Publication
(10) Pub. No.: US 2006/0270978 A1
(43) Pub. Date: Nov. 30, 2006

(54) Expandable esophageal access device

(76) Inventors: Ken Binmoeller, Ranch Santa Fe, CA (US); Matt Yurek, San Diego, CA (US)

Correspondence Address: SCOTT EVANS, 1252 COUNTRY HILLS DR., SANTA ANA, CA, 92705, US

(21) Appl. No.: 11/399,828

(22) Filed: Apr. 7, 2006

Related U.S. Application Data
(60) Provisional application No. 60/672,056, filed on Apr. 8, 2005.

(51) Int. Cl. A61M 29/00

(52) U.S. Cl. 604/104

(57) ABSTRACT

The present invention is directed to an apparatus and method for an expandable access device which includes an elongated braided tube having distal and proximal portions, the tube initially configured in a preformed radially compact configuration that is particularly sized for introduction into a body lumen. The access device has at least one deployment element that is attached to the distal portion of the braided tube and the tube is configured such that as the deployment element and the distal portion are pulled toward the proximal portion, the tube radially expands.

### CLAIM OF PRIORITY

This application claims priority under 35 U.S.C. § 119(e) to U.S. Provisional Application No. 60/672,056, filed Apr. 8, 2005, the entire contents of which are hereby expressly incorporated by reference.

### BACKGROUND

1. Field of the Invention

This invention relates to a system for delivering medical instruments to various body lumens but particularly the esophagus. The present invention relates generally to apparatus and methods for delivering instruments and/or agents during a medical procedure, and, more particularly, to guide sheaths for accessing body lumens and/or delivering instruments into body lumens of a patient, and to methods for making and using them

2. Description of the Related Art

Endoscopes and other accessory instruments are used to facilitate the expanding field of minimally invasive surgery. In these operations endoscopes are used to gain access to many internal lumens of the body including the upper gastrointestinal tract (esophagus, stomach and small bowel, pancreatic and bile ducts) through the mouth and the lower gastrointestinal tract (rectum and large bowel) through the anus. The introduction of these instruments can often be difficult for the operator and traumatic for the patient. This difficulty is sometimes caused by small passageways, tortuous anatomies and irritated or inflamed tissue. For example, the passage across restricted diameter portions of the esophagus, such as the cricopharynx, can be difficult due to the distal location of the restriction or an angulation or anatomical deformity often associated with discomfort and gagging that may result in tissue injury ranging from tears to perforation. A device that allows easier access and provides a safe channel into long body lumens like the trachea, colon and particularly the esophagus is needed. Furthermore, due to the increase in procedures performed through body orifices that require larger and stiffer instruments and repeated intubations, a flexible, expandable access device can improve comfort and safety for the patient.

Endoscopic overtubes are currently available and are made of plastic and have a fixed lumen diameter. During upper endoscopy, the overtube serves to protect the cricopharyngeal area and esophagus from trauma and the airway from aspiration. Passage of the overtube over the endoscope risks trauma and perforation due to its relative stiffness and the gap created between the endoscope and the overtube. This gap can entrap tissue during the process of sliding the overtube over the endoscope. To decrease this gap, the passage of the overtube is often performed without using the preferred method with an endoscope but is rather performed over an obturator called a bougie that has a distal end diameter that approximates the overtube. Because the overtube has a fixed, relatively large lumen diameter, the risk of trauma and perforation remains high due to the diameter and relative inflexibility of the overtube. Currently available overtubes have a fixed diameter that is the sum of their large inner lumen and the thickness of the tube wall sections. Whereas this size facilitates passing instruments through their inner lumen easily, the bulky size makes placement of the overtube itself difficult.

U.S. Pat. No. 5,460,170 to Julius G. Hammerslag, the entirety of which is hereby included by reference, discloses an adjustable surgical retractor incorporating the elements of a tubular mesh wherein the mesh is compressed axially to cause the retractor to expand radially. The axial compression is effected using a controller that rotates to wind up pull wires that are attached to the distal end of the tubular mesh causing axial compression and radial dilation of the sheath. However the Hammerslag device is designed to enlarge surgical openings in which the device is used to spread apart surgical incisions in the skin or other tissue. This tissue is typically close to the skin and thus the length of the device required for this application is relatively short. The uses and device described by Hammerslag would not be properly designed for use as an access sleeve, particularly one designed for the esophagus. The axial length of the tubular mesh, of Hammerslag in the radially expanded state is described as between 0.5 cm and 7 cm. This length is insufficient for effective use as an esophageal access device.

The expandable esophageal access device of the current invention is placed in the esophagus in a reduced diameter condition with a diameter that is close to that of the endoscope or obturator. This reduced size that is similar to the endoscope, reduces the gap between the device and the endoscope and the overall diameter of the system to ease introduction. The length of the device is also sized to insure that the access device is long enough to traverse the critical esophageal anatomies such as the cricopharynx. The flexible access device can then be then expanded after placement into the esophagus thereby creating a large flexible channel into the esophagus. One end is attached to a bite block in the mouth of the patient and the other freely resides in the esophagus, stomach, gastric tract or bowel. The esophageal access device can then be reduced in diameter when removal is indicated.

### BRIEF SUMMARY OF THE INVENTION

Accordingly the present invention is directed to an apparatus and method that is embodied and broadly described herein, where one aspect of the invention provides for an expandable access device which includes an elongated braided tube having distal and proximal portions, the tube initially configured in a pre-formed radially compact configuration that is particularly sized for introduction into a body lumen. The access device has at least one deployment element that is attached to the distal portion of the braided tube and the tube is configured such that as the deployment element and the distal portion are moved toward the proximal portion, the tube radially expands.

According to another embodiment of the present invention, the access device is an esophageal access device that may be used to protect the esophagus and facilitate the introduction of medical instruments such as an endoscope or surgical instruments into or through the esophagus.

In another aspect of the present invention the deployment element is a cord, string, rod or wire that extends from the distal portion of the tube to at least the proximal portion of the tube and typically to a position outside the patient's body where it can be manipulated by an operator to expand the braided tube. This deployment element may have sufficient column strength so that is may also be used to lengthen a previously compressed braided tube to reverse the radial expansion and reduce the braided tube cross sectional area for removal from the esophagus.

In still another embodiment of the present invention, the braided tube is comprised of a flexible polymeric braid. And in another embodiment the braided tube is made from a plastic alloy or a metal alloy. And in another embodiment the braided tube may be further comprised of a flexible covering disposed about the braided tube. And in another embodiment the braided tube may be further comprised of a flexible covering disposed inside the braided tube or the covering and the braid may be integrally formed.

In another embodiment of the invention, the braided tube may utilize a purse-string ligature that is disposed at the distal portion of the braided tube. The ligature is coupled to a pulling element to pull the purse-string ligature closed and collapse the diameter of the distal portion of the tube. This aspect may be used to secure the distal portion of the braided tube to an obturator or endoscope during insertion.

Another aspect of the invention, therefore, is to provide a method of introducing a medical instrument into an esophagus of a patient. The method includes: positioning the medical instrument into an elongated braided tube having a distal and a proximal portion; in which the tube may have a pre-formed radially compact configuration that is sized for introduction into a body lumen. The method also includes: collapsing a purse-string ligature around the distal portion of the medical instrument; inserting the elongated tube into the esophagus; releasing the purse-string ligature and radially expanding the elongated tube.

In another embodiment of the invention, the braided material or a covering disposed about the braid may be coated with various coatings that may aid in the insertion of the braided tube or the medical instrument or both. These coatings may be lubricious coatings or could possibly be pharmacologic or otherwise therapeutic in nature. These coatings and or substances may be applied to the surfaces of the tube or may be impregnated or admixed with the braid or covering.

All of these embodiments are intended to be within the scope of the present invention herein disclosed. These and other embodiments of the present invention will become readily apparent to those skilled in the art from the following detailed description of he preferred embodiments having reference to the attached figures. The invention is not limited to any particular preferred embodiment(s) disclosed.

### BRIEF DESCRIPTION OF THE DRAWINGS

FIG. 1 is a detail view of the esophageal access device shown in a collapsed condition;

FIG. 2a is a section view of the wall of the esophageal access device;

FIG. 2b is a section view of an alternate embodiment of the wall of the esophageal access device;

FIG. 2c is a section view of another alternate embodiment of the wall of the esophageal access device;

FIG. 3 is an end section view of the esophageal access device in a radially compact condition with an endoscope positioned inside;

FIG. 4 is a detail view of the esophageal access device shown in the esophagus of a patient;

FIG. 5 detail view of the esophageal access device shown partially expanded in the esophagus of a patient;

FIG. 6 is an end section view of the esophageal access device in an expanded condition with an endoscope positioned inside;

FIG. 7 is a detail view of the esophageal access device shown in an expanded condition;

FIG. 8 is a detail view of the of the distal end of the esophageal access device showing a purse string ligature;

FIG. 9 is a schematic view of an alternate embodiment of the esophageal access device shown inside a sheath.

### DETAILED DESCRIPTION OF THE INVENTION

The access device and method described herein may offer improvements over the techniques currently utilized to perform endoscopic procedures. The access device may be useful for providing a convenient method to introduce medical instruments to a body orifice. The device may be particularly useful when employing multiple devices or when an exchange of one device for another is necessary during long or difficult procedures. The access device may facilitate the introduction of said medical devices with less trauma or injury to the body orifice and may help in the introduction of large devices into a small orifice or an entry point with tortuous anatomies such as the cricopharynx in the esophagus.

The embodiments of this invention may be used in multiple locations in the body such as the esophagus, trachea or in internal body lumens such as the colon, intestines, pancreatic or bile ducts, or other vessels. Even though the descriptions in this application may focus on a particular application such as the esophagus, this description is not meant to be limiting and it is anticipated that the concept of an expandable access device may be useful in other lumens or vessels of the body as well.

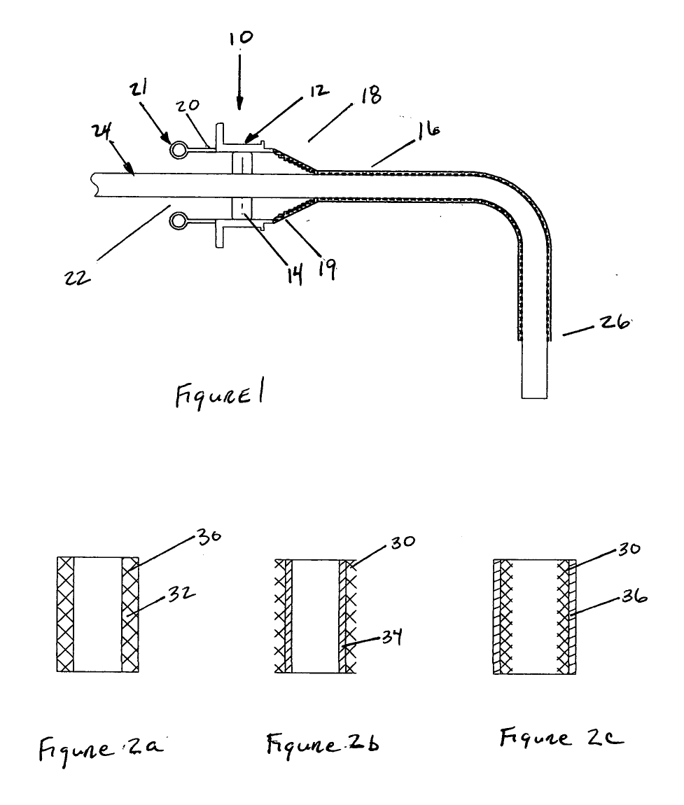

The access device 10 is comprised of a bite block 12 with a seal 14, a braided tube 16 with control element 20. The device is shown in FIG. 1 with an introducer, obturator or endoscope 24 positioned in the center of the tube. The access device 10 is in a compact condition suitable for introduction into the esophagus. In this condition the access device 10 provides a close fit to the outer diameter of an endoscope or obturator 24. The endoscope or obturator 24 can provide support and form to the access device 10 and this combination may facilitate it's insertion into the esophagus. However the access device 10 can also be constructed such that the device 10 has enough intrinsic support and rigidity to be inserted without the need for an endoscope or obturator. In this situation the access device 10 could be inserted over a guidewire directly into the esophagus. The proximal end 18 of the braided tube 16 is attached to the bite block 12 at point 19 in a manner so that the internal lumen transitions between the bite block 12 and the inner lumen of the braided tube are smooth.

The braided tube 16 comprises a braided material 30 that is flexible and capable of expansion and maintaining an open internal lumen despite placement around bends or anatomical curves. The braid 30 may utilize wires or ribbons that are designed to provide support for the esophageal access device's internal lumen but remain flexible to conform to the esophageal contours. The braid 30 may be constructed of plastic, metal or plastic or metal alloys. In addition to a braid the tube may be a woven mesh or net. A plastic braid can be formed from individual plastic strands that are woven into a braid with an initial diameter that can either self-expand or may expand when a force is applied to a second or larger diameter. The braid 30 may also be constructed from metallic ribbons or wires. The braid design and the manufacturing techniques useful for producing braided tubes of this type are commonly understood in the art (e.g. braids, weaves, knits, embroidery, etc). Likewise plastic or metal alloy wires, strands or ribbons that have superelastic or heat shapeable properties may be used. The braid 30 may employ braid geometries with various tensions, pitches or bias angles and even non braided strands are within the scope of the invention.

The braid 30 may be constructed as an expandable braid or a self-expanding braid. An expandable braid of this type is pre-formed with an initial smaller diameter and requires application of a force to expand the diameter to a larger diameter. This force is often a compression force that pulls the two ends of the braids together which has the effect of shortening the braided tube length and expanding the outer diameter. In this design the braid is important because as the braid length decreases the braid geometry resists collapse of the central lumen. In a preferred embodiment of the access device, the expansion force of the braid could be sufficient to enlarge an otherwise constricted lumen and provide a temporary lumen opening that has a diameter large enough for therapeutic procedures. The expandable braid may return to its original compact form once the compression force is released or the diameter may possibly be reduced by external geometries once the compression force is removed. Other expanding braids may require the application of a tension force to lengthen the braided length and reduce the larger diameter to a smaller diameter.

A self expanding braid does not require a compression force to increase the diameter from a smaller diameter to a larger one. This type of braid is formed with a diameter larger than the intended final diameter and is constrained to a much smaller diameter for insertion typically within an introducer sheath or axial tension on the braid. Once the introducer sheath is removed, this braid is designed to self expand until the constricting force of the body lumen, or by example the esophagus, equals the expansion force of the braid. The diameter of this type of braid can be reduced by repositioning the introducer sheath over the braid, axially extending the length of the braid with the control elements, an obturator or endoscope or by pulling on the proximal end of the mesh which reduces the braid diameter as it is pulled out of the body cavity.

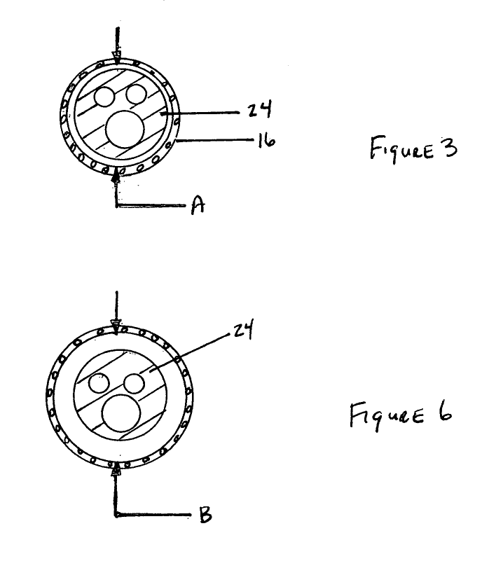

A covering may be disposed about the braided tube as shown in FIGS. 2a-c. This covering can be positioned on the outside or the inside of the braid. This covering may also be formed integrally to the braid itself. The covering is a flexible covering that can expand and contract as the braid expands and contracts. The covering encloses the openings between the braid strands and prevents fluids from leaking into or out of the braided tube. This may be important to prevent body fluids or air from leaking out through the walls of the braid. However in some situations the leakage of fluids may be less important and the covering may be unnecessary. The covering may be constructed from silicone, polyurethane, Kraton, latex or other plastic polymers. The covering can also be coated on its inner or outer surfaces with a lubricious coating to facilitate introduction, expansion and removal of the devices through the access device and of the access device to and from the body cavity itself Hydrophilic and hydrophobic coatings known in the art have been anticipated.

FIG. 2a shows a braided tube in which the braid 30 is imbedded in the covering 32. Alternatively, the braid 30 may surround a concentric expandable covering 34 and be attached to the outside of the braid as in FIG. 2b so that as the braid expands; the expandable covering 34 expands also. As shown in FIG. 2c, the braid 30 may also be surrounded concentrically by an expandable covering 36 with the braid 30 attached to the inside of the expandable covering 36. In any configuration it is important that the inner diameter of the braided tube 16 be smooth enough so as to facilitate the easy passage of endoscopes or instruments down the braided tube 16. In other words it is important that the inner surface be free of sharp points or large openings that could snag the passage of instruments down the braided tube 16 inner diameter. It is also important that the braided tube 16 be flexible so that the braid 30 can expand easily with minimal resistance.

The bite block 12 is the proximal terminus of the braided tube 16. The block is sized so that it can be placed between the teeth of the patient. The patient may bite down on this block to secure the access device 10 in the patient's esophagus and to insure that the device is positioned in the correct location. The control element 20 is used to apply an axial compression force on the distal end of the braided tube 16 to expand the tube 16. Although two control elements are shown in FIG. 1, it is expected that one or more control elements can be utilized. The control element 20 may be a flexible cord, string, wire or rod. In one embodiment the control element 20 is a nylon cord that is attached to a pull ring 21 at its proximal end. The control element 20 in this embodiment passes through a hole in the bite block 12 and is woven through the braid 30 of the braided tube 16 and secured at the distal end 26 of the braid 30. Alternately the control element 20 may pass along the outside or inside walls of the braided tube 16 or through a conduit formed in the walls of the braided tube 16. Even though the control element 20 may be located inside the braided tube 16 walls in some configurations, the control element 20 can freely move within, through or along the braided tube 16 walls.

In another embodiment the control element 20 is a stainless steel or Nitinol rod or wire that can be used to effect axial compression of the braided tube 16. The rod or wire described has column rigidity but is also flexible to negotiate the tortuous anatomy. The rod or wire is also useful in reversing the axial compression and radial expansion. Because the wire or rod is relatively stiff compared to a string for example, the rod or wire may be pushed as well as pulled so that a tension force can be applied to the distal portion of the braided tube. The tension force lengthens the tubular braid 16 and results in radial compression of the braid.

The seal 14 which is positioned inside the bite block 12 is designed to seal around the endoscope 24 or other instruments when they are positioned in the braided tube 16. This seal minimizes the leakage or back-flow of fluid, air or other biomaterials throughout the procedure and can be used to lock an instrument at a desired position within the body cavity. The seal adapts to the diameters of various sized medical instruments and may be a diaphragm with a central slit or an o-ring or compression type seal.

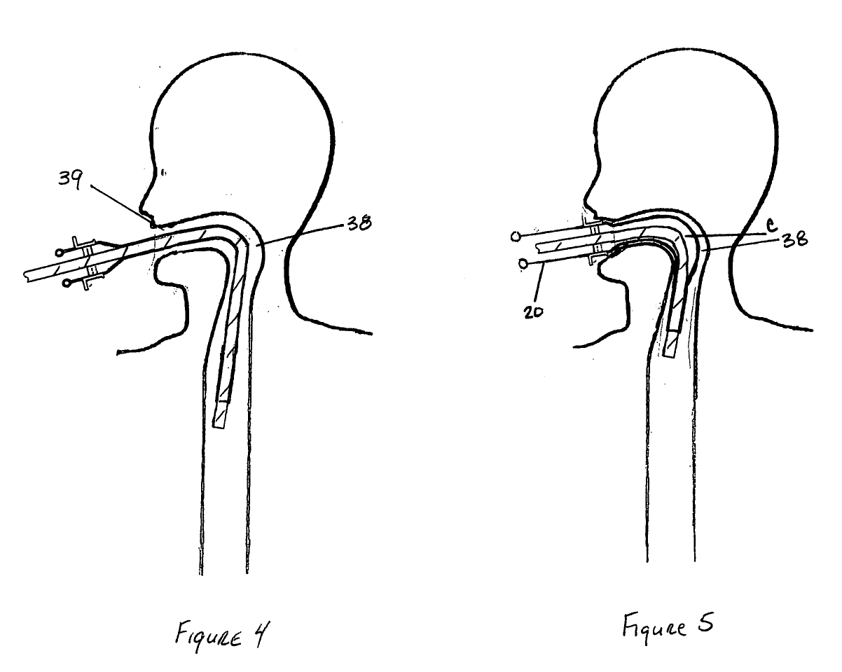

When used for esophageal access, the device 10 is prepared for introduction by inserting a guidewire into the esophagus. The obturator or endoscope 24 is brought to the proximal end 22 of the access device and passed through the seal 14 in the bite block 12, down the braided tube 16 and past the distal end of the braided tube 26. In this condition, the braided tube 16 is in a radially compact form having an inner diameter A as shown in FIG. 3, and is in close contact with the obturator or endoscope 24. The inner diameter of the braided tube in this configuration is preferably between 6 mm and 18 mm. This corresponds to the outside dimension of an endoscope which is typically 9 mm to 16 mm in diameter. At this point the inner diameter of the access device 10 and the outer diameter of the endoscope or obturator 24 are nearly identical.

The length of the access device 10 is sized for the particular anatomy intended. A longer length might be required for a lower or upper intestinal access device. In one preferred embodiment the access device is an esophageal access device and the length is sized for the esophageal anatomies. The esophagus is a tube that is particularly difficult to transverse because the tube makes a near 90 degree bend approximately 10 to 20 cm from the mouth. The cricopharynx, which is an esophageal sphincter, is located at this location and presents a particularly difficult portion of the anatomy to pass an endoscope or other medical instrument down the esophagus. The cricopharynx often creates a reduced diameter at this location and so any esophageal access device should extend beyond this structure. Accordingly the axial length of an esophageal access device in a radially expanded state needs to be at least longer than the location of the cricopharynx or at least 15-20 cm long. The axial length of the access device according to one aspect of the present invention in the radially expanded state is greater than 10 cm long. In a preferred embodiment the axial length of the access device in the radially expanded state is between about 20 cm and 35 cm long. In a particularly preferred embodiment the axial length of the access device in the radially expanded state is about 25 cm long.

The control element 20 is secured at the bite block 12 and the system is ready to be placed through the patient's mouth. The system is placed into the esophagus over the guidewire using standard techniques and initially positioned in the patient's mouth 39 as shown in FIG. 4. As shown, the device 10 is positioned in the patient's esophagus 38 with the bite block 12 outside the patient's mouth 39. Once the distal end 26 of the access device 10 is at the desired location (below the upper esophageal sphincter, in the lower esophagus, in the stomach or in the small bowel), the control element 20 is unsecured from the bite block 12 and held in a fixed position relative to the patient's mouth 39. The bite block 12 and proximal end 22 of the braid 16 are moved in a distal direction relative to the patient's mouth 39 until the bite block 12 is located at the teeth in the mouth 39.

Alternatively, the access device is placed over the endoscope or obturator and advanced into the esophagus until the bite block resides in the patient's mouth. The control element is then pulled proximally while the bite block is held within the teeth of the patient expanding the mesh within the esophagus.

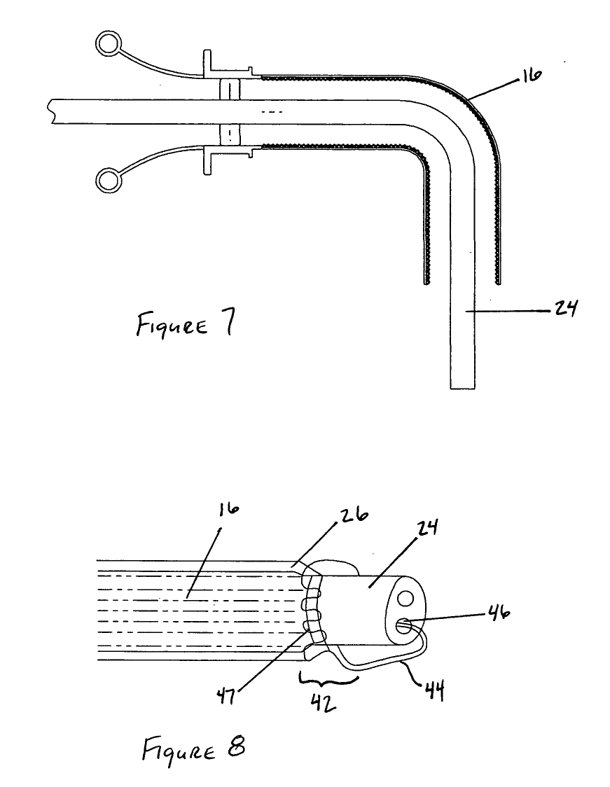

The access device 10 is shown in a partially expanded condition in FIG. 5. As shown, the position of the control element 20 is the same relative to its position in FIG. 4 as the bite block is moved in a distal axial direction into the mouth 39. In this figure, the proximal portion of the braided tube 16 is expanded inside the proximal portion of the esophagus 38. The gap C between the endoscope and the braided tube is evident at the rear of the patient's mouth. Once the braided tube 16 is fully expanded, the control element 20 is secured at the bite block 12 to maintain the condition of the braided tube 16. The distal end 26 of the braid is constrained from distal movement because the distal end 26 is attached to the distal end of the control element 20.

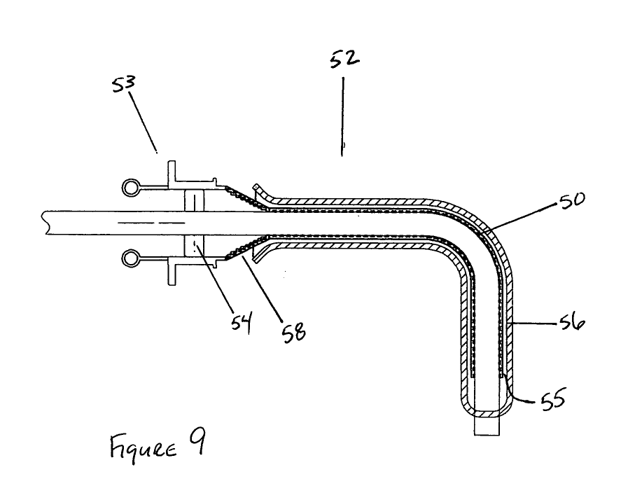

As shown in FIGS. 6 and 7, once fully expanded, the braided tube has expanded to a new radial diameter with a new inner diameter B that is larger than the outer diameter of the endoscope or obturator 24. The axial length of the braided tube has decreased and the radial diameter increased. In this expanded condition, at least one endoscope or other instrument may be passed down the access device 10 and into the digestive tract. The endoscope or obturator 24 can also be exchanged for other instruments.

The access device 10 can be removed by unsecuring the control element 20 from the bite block 12 and pulling the bite block 12 in a proximal direction while allowing the control elements 20 to move distally. This action removes the compression force, allows the braided tube 16 to relax which lengthens the braided tube 16 and decreases the inner diameter from diameter B to diameter A. The access system and endoscope can then be removed using conventional techniques.

In an alternate embodiment of the access device 10, a constraint element 42 may be utilized to temporarily constrain the distal end 26 of the braided tube 16 near the end of the endoscope 24 during insertion. This constraint element 42 is intended to prevent the distal end 26 of the braided tube 16 from sliding up the body of the endoscope or obturator 24 during insertion. This condition might occur when placing the access device 10 through narrow strictures or other reduced diameter areas of the esophagus or other areas of the body. As shown in FIG. 8, a constraint element 42 is formed at the distal end 26 of the braided tube 16. This constraint element 42 is formed with a ligature 44 that extends down the working lumen 46 of the endoscope 24 and is woven around the diameter of the distal end of the braided tube 16 in a purse string knot 47 and then is fed back up the working lumen 46 of the endoscope 24. When the access device 10 is inserted into the esophagus, both ends of the ligature 44 are pulled taught and the purse string knot 47 cinches tight against the body of the endoscope 24. This secures the distal end 26 of the braided tube 16 to the endoscope 24. When the access device 16 placement is completed, one end of the ligature 44 is pulled and the other end released, the purse string knot 47 relaxes and the ligature 44 is withdrawn through the working lumen 46 of the endoscope 24.

In another embodiment of the current invention the braided tube 50 is a self expanding type of braid and is biased toward the expanded condition. In this configuration once the braided tube 50 is allowed to expand, it may expand until it reaches a manufactured diameter or is restricted by the esophageal walls or esophageal sphincters. As shown in FIG. 9, this access device 52 has a braided tube 50 and a proximal assembly 53 which contains a seal 54. The access device 52 is initially constrained inside the inner lumen 55 of an outer sheath 56. The proximal assembly 53 and seal 54 are detachably connected to the access device 52. The sheath 56 constrains the expansion of the braided tube 50 to a diameter that is suitable for introduction and placement into an esophagus or other body lumen. Once the sheath 56 and access device 52 have been properly positioned inside the esophagus of a patient, the sheath 56 is slowly removed and the braided tube 50 expands to fill the esophageal space. The removal of the sheath 56 may be facilitated by attaching the proximal assembly 53 after the sheath has been withdrawn. Alternatively the sheath 56 may have a slit (not shown) formed longitudinally along one side that facilitates the removal of the sheath over the proximal end 58 of the braided tube 56 even in the presence of an attached proximal assembly 53. Sheaths of this type are known as “peel away” sheaths and are commonly used in interventional procedures.

Subsequently, the access device 52 can be removed by withdrawing the endoscope or other instrument and detaching the proximal assembly 53 from the braided tube 50. An outer sheath 56 is then slowly advanced distally over the braided tube 50 as the braided tube proximal end is held in place by the operator. Once the braided tube 50 is completely confined within the walls of the sheath 56, the braided tube 50 and the sheath 56 can be removed from the esophagus as a unit.

In another embodiment of the access device 10, the covering of the braid can also incorporate pharmaceuticals to affect the chemical responses of tissue, chemical processes or nerves adjacent to the braided tube. For example the braided tube may impregnated with drugs that may be useful to control the gag reflex, pain, saliva production or other biological or chemical conditions of the patient. Alternatively the braid or the covering may be coated with a lubricious coating that reduces friction on the inside or the outside of the braided tube. Such a lubricious coating might facilitate the smooth introduction of the braided tube into the body lumen and facilitate instrument introduction or exchange within the braided tube.

In another embodiment the access device is modified for use in the lower gastrointestinal tract to provide access to the colon (“endoscopic colon access device”). This device has a size modified for the colon and the anus and the bite block is replaced with a proximal assembly. A self expanding access device or an expandable device may be utilized in this application. Introduction and removal of this access device is similar to that previously described.

In still another embodiment, the access device is modified for use across a tracheostomy opening (“expandable tracheostomy device”). The length of the access device would be similar but the diameter would be smaller (4 to 10 mm). An airtight coating would need to surround or encapsulate the braid to allow connection to a ventilation system.

Although this invention has been disclosed in the context of certain preferred embodiments and examples, it will be understood by those skilled in the art that the present invention extends beyond the specifically disclosed embodiments and/or uses of the invention and obvious modifications and equivalents thereof. Thus it is intended that the scope of the present invention herein should not be limited by the particular disclosed embodiments described above, but should be determined only by a fair reading of the claims that follow.

What is claimed is:

1. An esophageal access device comprising:
an elongated braided tube having distal and proximal portions, the tube in a pre-formed radially compact configuration sized for introduction into a body lumen, at least one deployment element attached to the distal portion of the braided tube, the tube configured such that as the deployment element and the distal portion move toward the proximal portion, the tube radially expands.

2. The deployment element according to claim 1, whereby the deployment element is a cord, string, rod or wire and a proximal end of the deployment element extends outside a patient's body.

3. The deployment element of claim 2, wherein the deployment element is capable of transmitting a pulling or pushing force

4. The deployment element according to claim 3, wherein the deployment element is made of super elastic materials.

5. The esophageal access device according to claim 2, wherein the radially expanded braided tube has a length greater than 10 cm.

6. The esophageal access device according to claim 5, wherein the radially expanded braided tube has a length greater than 20 cm.

7. The esophageal access device according to claim 6, wherein the radially expanded braided tube has a length of between 20 cm and 40 cm.

8. The braided tube according to claim 2 whereby the braided tube is made of a flexible polymeric material.

9. The braided tube according to claim 2 whereby the braided tube is made of a flexible plastic or metal alloy.

10. The braided tube according to claim 2 in combination with an obturator or endoscope that is positioned inside the braided tube prior to introduction into the body lumen.

11. The braided tube according to claim 10, further comprising a purse-string ligature disposed at the distal portion of the tube, said ligature coupled to a pulling element to pull the purse-string ligature closed and collapse the diameter of the distal portion of the tube thereby securing the distal portion of the braided tube to the obturator or endoscope.

12. The braided tube according to claim 10, further comprising a flexible covering disposed about the braided tube.

13. The tube according to claim 2 whereby the tube is coated with a lubricious coating that aids in the delivery of the tube into the esophagus.

14. The tube according to claim 2 whereby the tube is coated or impregnated with a pharmacological or therapeutic agent.

15. The esophageal access device according to claim 11 further comprising a block coupled to a proximal end of the braided tube, the block having a through lumen in communication with the lumen of the tube, the block sized for placement in between the patient's teeth, and the block having a seal or lock positioned inside to seal around the obturator or endoscope positioned inside the tube.

16. A body lumen access device comprising:
an elongated braided tube having distal and proximal portions, the tube pre-formed in a radially compact configuration sized for introduction into a body lumen,
at least one deployment element attached to the distal portion of the braided tube, the tube configured such that as the deployment element and the distal portion of the braided tube move toward the proximal portion with the position of the proximal portion being fixed, the tube is expanded radially.

17. The deployment element according to claim 16, whereby the deployment element is a cord, string or wire and a proximal end of the deployment element extends outside a patient's body.

18. The deployment element of claim 17, wherein the deployment element is capable of transmitting a pulling or pushing force.

19. The deployment element according to claim 18, wherein the deployment element is made of super elastic materials.

20. The esophageal access device according to claim 17, wherein the radially expanded braided tube has a length greater than 10 cm.

21. The esophageal access device according to claim 20, wherein the radially expanded braided tube has a length greater than 20 cm.

22. The esophageal access device according to claim 21, wherein the radially expanded braided tube has a length of between 20 cm and 40 cm.

23. The braided tube according to claim 17 whereby the braided tube is made of a flexible polymeric material.

24. The braided tube according to claim 17 whereby the braided tube is made of a flexible plastic or metal alloy.

25. The tube according to claim 17 in combination with an obturator or endoscope that is positioned inside the braided tube prior to introduction into the body lumen.

26. The braided tube according to claim 25, further comprising a purse-string ligature disposed at the distal portion of the tube, said ligature coupled to a pulling element to pull the purse-string ligature closed and collapse the diameter of the distal portion of the tube thereby securing the distal portion of the braided tube to the obturator or endoscope.

27. The braided tube according to claim 25, further comprising a flexible covering disposed about the braided tube.

28. The tube according to claim 17 whereby the tube is coated with a coating that aids in the delivery of the tube into the esophagus.

29. The tube according to claim 17 whereby the tube is coated or impregnated with a pharmacological or therapeutic agent.

30. The esophageal access device according to claim 26 further comprising a block coupled to a proximal end of the braided tube, the block having a through lumen in communication with the lumen of the tube, the block sized to be placed in between the patient's teeth, and the block having a seal or lock positioned inside to seal around the obturator or endoscope positioned inside the tube.

31. A method of introducing an elongated medical instrument into a body lumen of a patient comprising the steps of:
positioning the medical instrument into an elongated braided tube having distal and proximal portions, the tube in a pre-formed radially compact configuration sized for introduction into a body lumen, collapsing a purse-string ligature around the distal portion of the medical instrument, inserting the elongated tube into the body lumen, releasing the purse-string ligature and radially expanding the elongated tube.

32. The method according to claim 31, wherein the expanding step further comprises pulling on a deployment element that is attached to the distal portion of the tube such that as the deployment element and the distal portion of the braided tube are moved toward the proximal portion, the tube is expanded in a radial direction.

33. A method of introducing a medical instrument into an esophagus comprising the steps of:
introducing a protective braided sleeve into the esophagus, the sleeve having a first pre-formed radially compact configuration, radially expanding the sleeve to a second configuration having an expanded diameter compared to the first configuration, and;
introducing a medical instrument into the esophagus through the sleeve.

34. The method according to claim 33 wherein the expanding step comprises pulling a deployment element coupled to the distal portion of the sleeve such that as the deployment element and the distal portion of the braided tube are moved toward the proximal portion, the tube radially expands.

35. The method according to claim 34 further comprising positioning a block that is coupled to a proximal end of the sleeve in between a patient's teeth and causing the patient to bite the block to secure the position of the proximal end of the sleeve.

36. The method according to claim 35 further comprising removing the medical instrument and releasing the deployment element thereby allowing the sleeve to collapse to a more radially compact configuration.

37. The method according to claim 35 further comprising removing the medical instrument from the sheath and pushing the deployment element to lengthen the braided sleeve and reduce the outer diameter of the sleeve, and removing the sleeve from the esophagus.
